(19) United States
(12) Patent Application Publication
(10) Pub. No.: US 2012/0308663 A1
(43) Pub. Date: Dec. 6, 2012

(54) LIPID NANOCAPSULES, METHOD FOR PREPARING SAME AND USE THEREOF AS A DRUG

(76) Inventors: Emilie Roger, Angers (FR); Frederic Lagarce, Angers (FR); Jean-Pierre Benoit, Angers (FR)

(21) Appl. No.: 13/497,914

(22) PCT Filed: Sep. 24, 2010
(86) PCT No.: PCT/EP10/64102

§ 371 Date: Aug. 20, 2012

(30) Foreign Application Priority Data
Sep. 24, 2009 (FR) 0956585

(51) Int. Cl. A61K 9/51; A61K 9/127; A61K 31/4745; A61P 35/00; B82Y 40/00; B82Y 5/00

(52) U.S. Cl. 424/502; 514/283; 264/4.1; 977/798; 977/906; 977/900

(57) ABSTRACT

The present invention relates to nanocapsules, including: a core essentially consisting of a fatty substance, which is liquid or semi-liquid at ambient temperature, and including a hydrophobic active principle and a diethylene glycol ether; an outer lipid shell which is solid at ambient temperature. The lipid nanocapsules of the invention are intended in particular for the manufacture of a drug.

The object of the present invention is lipid nanocapsules (LNCs), a method for their preparation and their use for making a drug, notably intended to be administered orally.

These recent years, many groups have developed formulations of lipid solid nanoparticles or lipid nanospheres (Müller, R. H. et Mehnert, European Journal of Pharmaceutics and Biopharmaceutics, 41(1): 62-69, 1995; W., Gasco, M. R., Pharmaceutical Technology Europe: 52-57, December 1997; EP 605 497). This is an alternative to the use of liposomes or polymer particles. These lipid particles have the advantage of being formulated in the absence of a solvent. They have allowed encapsulation of both lipophilic and hydrophilic products in the form of ion pairs for example (Cavalli, R. et al. S.T.P. Pharma Sciences, 2(6): 514-518, 1992; and Cavalli, R. et al. International Journal of Pharmaceutics, 117: 243-246, 1995). These particles may be stable over several years away from light, at 8° C. (Freitas, C. et Müller, R. H., Journal of Microencapsulation, 1 (16): 59-71, 1999).

Two techniques are currently used for preparing lipid nanoparticles:
homogenization of a hot emulsion (Schwarz, C. et al. Journal of Controlled Release, 30: 83-96, 1994; Müller, R. H. et al. European Journal of Pharmaceutics and Biopharmaceutics, 41(1): 62-69, 1995) or a cold emulsion (Zur Mühlen, A. and Mehnert W., Pharmazie, 53: 552-555, 1998; EP 605 497), or
chill-hardening of a micro-emulsion in the presence of co-surfactants such as butanol. The size of the obtained nanoparticles is generally greater than 100 nm (Cavalli, R. et al. European Journal of Pharmaceutics and Biopharmaceutics, 43(2): 110-115, 1996; Morel, S. et al, International Journal of Pharmaceutics, 132: 259-261, 1996). Cavalli et al. (International Journal of Pharmaceutics, 2(6): 514-518, 1992; and Pharmazie, 53: 392-396, 1998) describe the use of a biliary salt, taurodeoxycholate, non-toxic by injection for forming nanospheres with a size greater than or equal to 55 nm.

The Applicant in International Application WO01/64328 discloses nanocapsules consisting of a liquid or semi-liquid core at room temperature, coated with a solid film at room temperature. In particular, the core of these nanocapsules essentially consists of a liquid or semi-liquid fat at room temperature, for example a triglyceride or a fatty acid ester, and the solid film coating the nanocapsules essentially consists of a lipophilic surfactant, for example a lecithin, for which the proportion of phosphatidylcholine is comprised between 40 and 80%. The preparation method is based on a step of phase inversion of the oil/water emulsion formed by the constituents of the nanocapsules.

However, this method requires solubilization of the active ingredient. Now, certain active ingredients are insufficiently soluble in a fatty phase in order to be encapsulated according to this method.

The technical problem solved by the present invention consists of proposing a method for preparing nanocapsules which allows encapsulation into nanocapsules of a sufficient amount of such active ingredients.

Unexpectedly, the Applicant developed such a method. The inventors have in particular observed that the solubilization of such an active ingredient in a diethylene glycol ether is required for solving the technical problem.

The present invention therefore relates to nanocapsules comprising:

a core essentially consisting of a liquid or semi-liquid fat at room temperature, and comprising a hydrophobic active ingredient and a diethylene glycol ether,

an external solid lipid shell at room temperature.

By nanocapsules are meant particles consisting of a liquid or semi-liquid core at room temperature, coated with a solid film at room temperature, as opposed to nanospheres which are matrix particles, i.e. for which the totality of the mass is solid. When the nanospheres contain a pharmaceutically active ingredient, the latter is finely dispersed in the solid matrix.

Within the scope of the present invention, by room temperature is meant a temperature comprised between 15 and 25° C.

The object of the present invention is nanocapsules with an average size of less than 150 nm, preferably less than 100 nm, still preferably less than 50 nm.

Considering their size, the nanocapsules of the invention are colloidal lipid particles.

The polydispersity index of the nanocapsules of the invention is advantageously comprised between 5 and 15%.

Preferably, the diethylene glycol ether used is selected from the group formed by diethylene glycol monoethyl ether, diethylene glycol monomethyl ether, diethylene glycol mono-n-butyl ether.

More preferably, diethylene glycol monoethyl ether, for example Transcutol® HP is used as a diethylene glycol ether.

The active ingredient is either not or not very soluble or dispersible in an oily fat phase and preferably is not soluble in most pharmaceutically acceptable solvents.

Advantageously, the active ingredient is SN38.

SN38, 7-ethyl-10-hydroxycamptothecin is the active metabolite of Irinotecan (CPT11), an inhibitor of topoisomerase I. SN 38 is 200 to 2,000 times more toxic than CPT11. However, SN38 has not been used as anticancer agent because of its low solubility in pharmaceutically acceptable solvents.

Advantageously, the nanocapsules according to the invention contain more than 0.1 mg, preferably 0.3 mg, more preferably 0.4 mg of active ingredient per gram of nanocapsules.

Preferably, the fat of the core essentially consists of at least one triglyceride, a fatty acid ester, a polyethoxylene glyceride or one of their mixtures.

The fat of the core accounts for 20 to 60%, preferentially 25 to 50% by weight of the nanocapsules.

The applied triglycerides may be synthetic triglycerides or triglycerides of natural origin. The natural sources may include animal fats or vegetable oils for example soyabean oils or long chain triglyceride sources (LCT).

Other triglycerides of interest mainly consist of fatty acids with medium lengths, further called medium chain triglycerides (MCT). An oil with medium chain triglycerides (MCT) is a triglyceride in which the hydrocarbon chain has from 8 to 12 carbon atoms (C₈-C₁₂). Such MCT oils are available commercially.

As an example of these MCT oils, mention may be made of TCR (tradename of the “Society Industrielle des Oléagineux”, France, for a mixture of triglycerides in which about 95% of the fatty acid chains have 8 or 10 carbon atoms) and Miglyol 812 (a triglyceride marketed by Dynamit Nobel and Sasol Condea Chemie, for a mixture of caprylic and capric acid glyceride triesters).

The fatty acid units of these triglycerides may be unsaturated, mono-unsaturated or poly-unsaturated. Mixtures of triglycerides having variable fatty acid units are also acceptable.

The triglyceride making up the core of the nanocapsules is notably selected from C₈-C₁₂ triglycerides, for example triglycerides of capric and caprylic acids and mixtures thereof.

The fatty acid ester is selected from C₈-C₁₈ fatty acid esters, for example ethyl palmitate, ethyl oleate, ethyl myristate, isopropyl myristate, octydodecyl myristate, and their mixtures. The fatty acid ester is preferably a C₈-C₁₂ fatty acid ester.

The polyethoxylene glyceride is selected from a mixture of glycerides and polyethylene glycol, a PEG-6 ester and of apricot kernel oil, olive oil, hydrogenated palm oil for example Labrafil® M 1944 CS, Labrafil® M 1969 or Labrafil® M 1980 (Gattefossé, Saint Priest, France).

Advantageously, the external shell of the nanocapsules according to the invention essentially consists in a lipophilic surfactant and a hydrophilic surfactant.

Preferably, the lipophilic surfactant is a phospholipid such as lecithins, phosphatidylglycerol, phosphatidylinositol, phosphatidylserine, phosphatidic acid and phosphatidylethanolamine.

Phospholipids are advantageous because of their biocompatibility.

As commercial products derived from phospholipids, mention may more particularly be made of:
EPICURON 120 (Lukas Meyer, Germany) which is a mixture of about 70% of phosphatidylcholine, 12% of phosphatidylethanolamine, and about 15% of other phospholipids;
OVOTINE 160 (Lukas Meyer, Germany) which is a mixture comprising about 60% of phosphatidylcholine, 18% of phosphatidylethanolamine, and 12% of other phospholipids,
a mixture of purified phospholipids reflecting the Lipoid E75 or Lipoids E-80 products (Lipoid, Germany) which is a mixture of phospholipids comprising about 80% by weight of phosphatidylcholine, 8% by weight of phosphatidylethanolamine, 3.6% by weight of non-polar lipids and 2% of sphingomyelin.

According to a preferred embodiment, the lipophilic surfactant is a lecithin, for which the proportion of phosphatidylcholine varies from 40 to 80% by weight. Lipoid S75-3 (Lipoid GmbH, Germany) is most particularly suitable as a source of phosphatidylcholine. This is a soya lecithin. The latter contains about 69% of phosphatidylcholine and 9% of phosphatidylethanolamine. This constituent is the only solid constituent at 37° C. and at room temperature in the formulation. It is possible to use polyglyceryl-6-dioleate (Plurol®).

The hydrophilic surfactant applied according to the present invention is advantageously an amphiphilic hydrophilic surfactant.

The emulsifying surfactants of the oil-in-water type customarily used have an HLB (HLB=Hydrophilic Lipophilic Balance) ranging from 8 to 18. These emulsifiers by their amphiphilic structure are located at the oil phase/aqueous phase interface, and thus stabilize the dispersed oil droplets.

Thus, the surfactant system used in the micro-emulsion may comprise one or more surfactants, the solubility of which in oil increases with increasing temperature. The HLB of the surfactants may vary from 8 to 18 and preferably from 10 to 16, and the surfactants may be selected from ethoxylated fatty alcohols, ethoxylated fatty acids, ethoxylated fatty acid partial glycerides, polyethoxylated fatty acid triglycerides and mixtures thereof. As ethoxylated fatty alcohols, mention may for example be made of adducts of ethylene oxide with lauryl alcohol, notably those including from 9 to 50 oxyethylene groups (Laureth-9 to Laureth-50 in CTFA names); adducts of ethylene oxide with behenyl alcohol, notably those including from 9 to 50 oxyethylene groups (Beheneth-9 to Beheneth-50 in CTFA names); adducts of ethylene oxide with cetostearyl alcohol (a mixture of cetyl alcohol and stearyl alcohol), notably those including from 9 to 30 oxyethylene groups (Ceteareth-9 to Ceteareth-30 in CTFA names); adducts of ethylene oxide with cetyl alcohol, notably those including from 9 to 30 oxyethylene groups (Ceteth-9 to Ceteth-30 in CTFA names); adducts of ethylene oxide with stearyl alcohol, notably those including from 9 to 30 oxyethylene groups (Steareth-9 to Steareth-30 in CTFA names); adducts of ethylene oxide with isostearyl alcohol, notably those including from 9 to 50 oxyethylene groups (Isosteareth-9 to Isosteareth-50 in CTFA names); and mixtures thereof.

As ethoxylated fatty acids, mention may for example be made of addition products of ethylene oxide with lauric, palmitic, stearic or behenic acids, and mixtures thereof, notably those including from 9 to 50 oxyethylene groups such as PEG-9 to PEG-50 laurates; (CTFA names: PEG-9 laurate to PEG-50 laurate); PEG-9 to PEG-50 palmitates (CTFA names: PEG-9 palmitate to PEG-50 palmitate); PEG-9 to PEG-50 stearates (CTFA names: PEG-9 stearate to PEG-50 stearate); PEG-9 to PEG-50 palmitostearates; PEG-9 to PEG-50 behenates; (CTFA names: PEG-9 behenate to PEG-50 behenate); and mixtures thereof.

It is also possible to use mixtures of these oxyethylene derivatives of fatty alcohols and of fatty acids. These surfactants may also be either natural compounds reflecting echolate phospholipids or synthetic compounds such as polysorbates which are polyethoxylated fatty acid esters of sorbitol (Tween®), fatty acid polyethylene glycol esters derived for example from castor oil (Cremophor®), polyethoxylated fatty acids, for example of stearic acid (Simulsol®M-53), polyoxyethylene fatty alcohol ethers (Brij®), polyoxyethylene non-phenyl ethers (Triton®N), polyoxyethylene hydroxylphenyl ether esters (Triton®X).

This may be more particularly a polyethylene glycol 2-hydroxystearate and notably the one marketed under the name of Solutol® HS15 by BASF (Germany).

The hydrophilic surfactant contained in the solid film coating the nanocapsules preferably accounts for between 20 to 50% by weight of the nanocapsules, preferably about 30%.

The amount of lipophilic surfactant contained in the solid film coating the nanocapsules of the invention is preferably set so that the liquid fat/solid surfactant compound mass ratio is selected between 1 and 15, preferably between 1.5 and 13, more preferentially between 3 and 8.

The nanocapsules of the invention are particularly adapted to the formulation of pharmaceutical active ingredients. In this case, the lipophilic surfactant may advantageously be solid at 20° C. and liquid at about 37° C.

Advantageously, the nanocapsules of the invention have a lipids/[active ingredient+diethylene glycol ether] core ratio comprised between 0.5:1 and 1:2.

Advantageously, the nanocapsules of the invention comprise
a core consisting of SN38, of diethylene glycol monoethyl ether, of a capric and caprylic triglyceride and of a PEG-6 ester of apricot kernel oil,
an external shell consisting of lecithin, preferably a lecithin for which the proportion of phosphatidylcholine is comprised between 40 and 90%, and of polyethylene glycol 2-hydroxystearate in a ratio from 1:0.09 to 0.15:1.

Advantageously, the core of the nanocapsules of the invention consist in
a central core consisting of the hydrophobic active ingredient and of a diethylene glycol ether,
a lipid layer consisting of the fats surrounding said core.

The object of the present invention is also a method for preparing the nanocapsules described earlier.

The method of the invention is based on phase inversion of an oil/water emulsion caused by several cycles of increasing and decreasing temperature.

The preparation method of the invention comprises the following steps:
a) solubilizing the active ingredient in a solution of diethylene glycol ether,
b) preparing an oil/water emulsion by adding to the solution of step a) at least one triglyceride, a polyethoxylene glyceride, a lipophilic surfactant solid at 20° C., a non-ionic hydrophilic surfactant, an aqueous phase and a salt,
c) achieving phase inversion of said oil/water emulsion by increasing the phase inversion temperature (PIT) with stirring, in order to obtain a water/oil emulsion, followed by decreasing the temperature down to a temperature T1, T1<PIT<T2,
d) carrying out one or several temperature cycles with stirring, preferably at least 3, around the phase inversion zone between T1 and T2, until a translucent suspension is observed,
e) achieving chill-hardening with an aqueous solution of the oil/water emulsion at a temperature close to T1, preferably greater than T1, in order to obtain stable nanocapsules.

With this step it is possible to stabilize the formed nanocapsules. It consists in sudden cooling, with magnetic stirring, by diluting the emulsion between 3 and 10 times with deionized water or with an acid buffer at 2° C.+/−1° C. thrown into the fine emulsion.

Thus, the whole of the constituents intended to form the emulsion is weighed in a container. The mixture is homogenized and heated, for example by means of stirring produced on a heating plate, up to a temperature greater than or equal to the phase inversion temperature T2, i.e. until a white phase is obtained which indicates that the inverse emulsion (W/O) has been obtained. Heating is then stopped and the stirring is continued until return to room temperature, by passing through the phase inversion temperature T1, i.e. the temperature at which the expected oil/water emulsion is formed, as a transparent or translucent phase. When the temperature has been lowered below the phase inversion temperature (PIT), an oil/water emulsion is obtained. More specifically, the phase inversion between the oil/water emulsion and the water/oil emulsion is expressed by a decrease in conductivity when the temperature increases. Thus, T1 is a temperature at which the conductivity is at least equal to 90-95% of the measured conductivity and T2 is the temperature at which the conductivity decreases and the water-in-oil emulsion is formed. The average temperature of the phase inversion zone corresponds to the phase inversion temperature (PIT). The organization of the system in the form of nanocapsules is visually expressed by a change in the aspect of the initial system which passes from opaque white to translucent white. This change occurs at a temperature below PIT. This temperature is generally located, 6 to 15° C. below PIT. In the zone for forming an oil/water emulsion (translucent mixture), the hydrophilic and hydrophobic interactions are balanced. By heating beyond this zone, there is formation of a W/O emulsion (opaque white mixture), since the surfactant promotes formation of a water-in-oil emulsion. Then, during the cooling below the phase inversion zone, the emulsion becomes an O/W emulsion.

The temperature T1 is comprised between 55 and 70° C., preferably between 60 and 70° C., more preferably T1 is 65° C.

The temperature T2 is comprised between 85 and 100° C., preferably between 85 and 95° C. More preferably T2 is 90° C.

The number of cycles applied to the emulsion depends on the amount of energy required for forming the nanocapsules. More preferably 3.

Advantageously, step b) is broken down as follows:

b1) adding to the solution of step a) at least one triglyceride, a polyethoxylene glyceride and a lipophilic surfactant,

b2) heating until solubilization of the lipophilic surfactant,

b3) cooling,

b4) adding the hydrophilic surfactant, the aqueous phase and the salt.

The heating step b2) is carried out a temperature comprised between 50 and 70° C., preferably 70° C., in particular when the lipophilic surfactant is a lecithin.

When the active ingredient is SN38, a basic buffer is added to step b4) for transforming SN38 as a free lactone into SN38 as a carboxylate and step e) of the method of the invention is carried out with an acid aqueous solution. When the active ingredient is SN38, the chill-hardening is carried out with an acid buffer in order to retransform SN38 into a lactone form at the moment of its encapsulation, preferably with an acid buffer at 2° C.±1° C.

The obtained particles after chill-hardening are maintained with stirring for 5 mins.

The nanocapsules obtained according to the method of the invention are advantageously without any co-surfactant agents, such as C₁-C₄ alcohols.

The oil/water emulsion advantageously contains 1 to 3% of lipophilic surfactant, 5 to 15% of hydrophilic surfactant, 5 to 10% of co-surfactant (diethylene glycol ether), 5 to 15% of oily fats, 40 to 65% of water (the percentages are expressed by weight).

The higher the HLB index of the liquid or semi-liquid fat, the higher is the phase inversion temperature. On the other hand, the value of the HLB index of the fat does not seem to have any influence on the size of the nanocapsules.

Thus, when the size of the terminal groups of the triglycerides increases, their HLB index decreases and the phase inversion temperature decreases.

The HLB or hydrophilic-lipophilic balance index is as defined by C. Larpent in the Traité K.342 of the Editions of the Techniques de I′lngénieur.

The size of the particles decreases when the proportion of hydrophilic surfactant increases and when the proportion of (hydrophilic and lipophilic) surfactants increases. Indeed, the surfactant causes a decrease in the interfacial tension and therefore a stabilization of the system which promotes the obtaining of small particles.

Moreover, the size of the particles increases when the oil proportion increases.

According to a preferred embodiment, the fat consists of Labrafac® WL 1349 and of Labrafil® M 1944 CS, the lipophilic surfactant is Lipoid® S 75-3 and the non-ionic hydrophilic surfactant is Solutol® HS 15 and the co-surfactant or solubilizing agent is Transcutol®HP. These compounds have the following characteristics:

Lipophilic Labrafac® WL 1349 (Gattefossé, Saint-Priest, France). This is an oil consisting of a caprylic and capric acid (C₈ and C₁₀) medium chain triglyceride. Its density is from 0.930 to 0.960 at 20° C. Its HLB index is of the order of 1.

Labrafill® M 1944 CS (Gattefossé, Saint-Priest, France).

This is an oleic macrogol glyceride (hydrophilic oil) consisting of mono-, di-, tri-glycerides and of fatty acid polyethylene glycol mono-, and di-esters. Its density is from 0.935 to 0.955 at 20° C. It is usable orally (in rats, DL 50>20 mL/kg).

Lipoid® S 75-3 (Lipoid GmbH, Ludwigshafen, Germany). Lipoid® S 75-3 corresponds to a soya lecithin. The latter contains about 69% of phosphatidylcholine and 9% de phosphatidylethanolamine. These are therefore surfactant compounds. This constituent is the only solid constituent at 37° C. and at room temperature in the formulation. It is commonly used for formulating injectable particles.

Solutol® HS 15 (BASF, Ludwigshafen, Germany). This is a polyethyleneglycol-660 2-hydroxystearate. It therefore plays the role of a non-ionic hydrophilic surfactant in the formulation. It may be used orally (in mice, intravenously DL50>3.16 g/kg, in rats 1.0<DL 50<1.47 g/kg).

Transcutol®HP (Gattefossé, Saint-Priest, France). This is a diethyleneglycol monoethyl ether. Its density is from 0.985 to 0.991 at 20° C. It plays the role of a solubilizing or co-surfactant agent. It may be used orally (in rats DL50>5 g/kg).

The aqueous phase of the oil/water emulsion may also contain 1 to 4% of a salt such as sodium chloride. Modification of the salt concentration causes a shift of the phase inversion zone. The more the salt concentration increases and the lower is the phase inversion temperature. This phenomenon will be of interest for encapsulation of hydrophobic heat-sensitive active ingredients. Their incorporation may be accomplished at a lower temperature.

The object of the present invention is also the nanocapsules of the invention for their use as a drug.

The object of the present invention is also the nanocapsules of the invention loaded with SN38 for treating cancer.

The present invention therefore also relates to the use of the nanocapsules of the invention for making a drug.

The present invention therefore also relates to the use of the nanocapsules of the invention loaded with SN38 for making a drug intended for treating cancer.

The present invention also relates to a treatment method comprising the administration of an effective amount of nanocapsules of the invention to a patient in need thereof.

The present invention also relates to a treatment method comprising the administration of an effective amount of nanocapsules of the invention loaded with SN38 to a patient affected with cancer.

The object of the present invention is also a pharmaceutical composition comprising nanocapsules of the invention and at least one pharmaceutically acceptable carrier.

In the present invention, the intention is to designate by <<pharmaceutically acceptable>> what is useful in the preparation of a pharmaceutical composition which is generally safe, non-toxic and neither biologically nor otherwise undesirable and which is acceptable for veterinary use as well as for human pharmaceutical use.

The pharmaceutical compositions according to the invention may be formulated for oral, sublingual, sub-cutaneous, intramuscular, intravenous, intrathecal, epidural, transdermal, local or rectal, preferably oral administration intended for mammals, including humans.

The nanocapsules of the invention may be freeze-dried. In this case, a cryoprotective agent such as trehalose may be added to the formulation in order to prevent aggregation of the nanoparticles and to maintain their redispersion. With freeze-drying, it is possible to improve the stability of the nanocapsules over time, and to also contemplate a dry formulation of these particles.

The nanocapsules of the invention may be administered as single dosage administration forms, mixed with conventional pharmaceutical supports, to animals or human beings. The suitable single dosage administration forms comprise the oral forms such as tablets, gelatin capsules, powders, granules and oral solutions or suspensions, sublingual and buccal administration forms, subcutaneous, intramuscular, intravenous, intranasal or intraocular administration forms and rectal administration forms.

The object of the present invention is also a pharmaceutical composition according to the invention as defined earlier for its use as a drug.

More particularly, the nanocapsules of the invention are suitable for administration of the following active ingredients:
anti-infectious agents among which are antimycotic agents, antibiotics,
anticancer agents,
active ingredients intended for the central nervous system (CNS), which have to pass through the blood-brain barrier, such as antiparkinson agents and more generally active ingredients for treating neurodegenerative diseases.

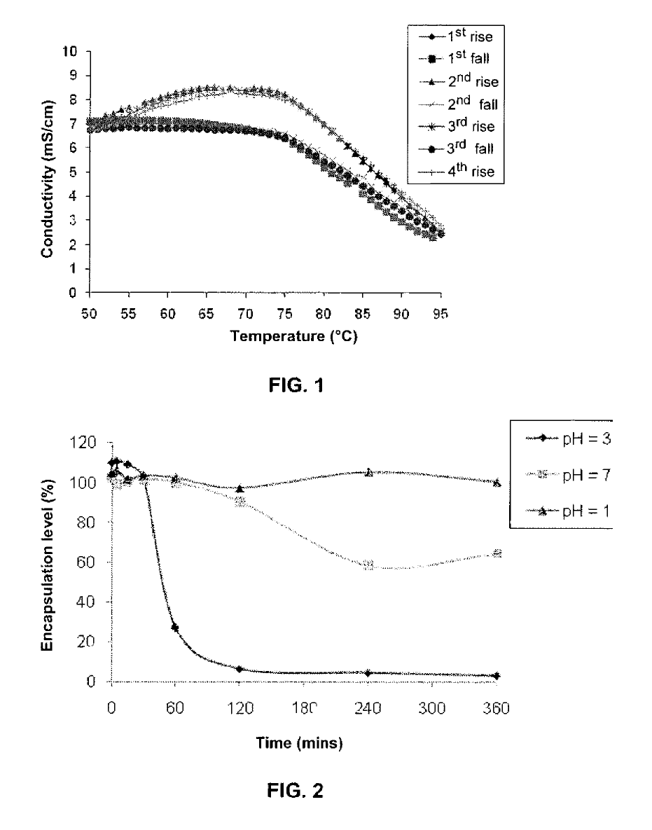

The present invention is illustrated by the following examples with reference to FIGS. 1 to 7.

FIG. 1 illustrates the time-dependent change in conductivity versus the temperature of the oil/water emulsion described in Example 2.

FIG. 2 illustrates the time-dependent change in the SN38 encapsulation level versus time for different pHs. 100% corresponds to the initial encapsulation level of the SN38 formulation.

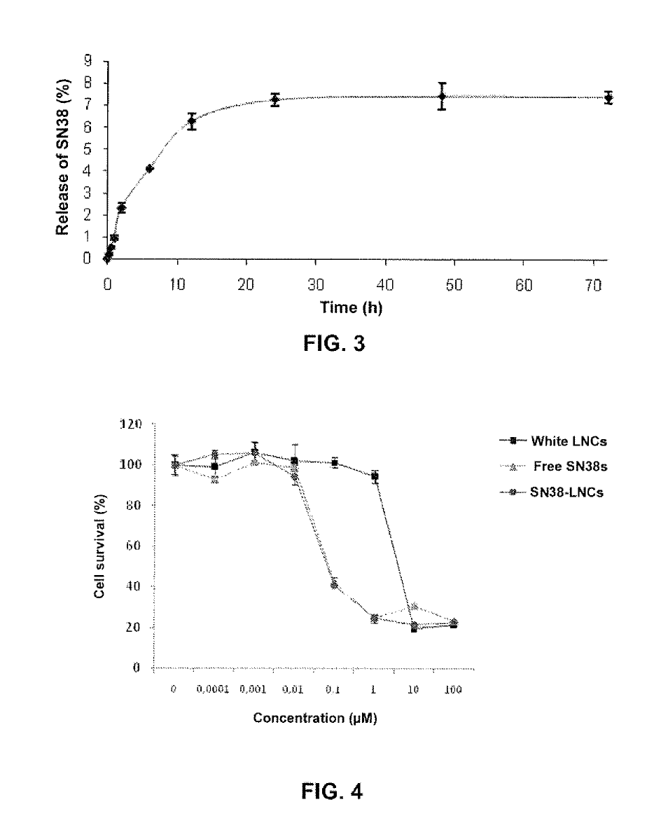

FIG. 3 illustrates the change in the SN38 release percentage from the initial encapsulation level versus time after storage of the formulation at 2-8° C.

FIG. 4 illustrates the cell survival percentage (HT-29 cells) versus the concentration of SN38-LNCs, SN38 or non-loaded LNCs (white LNCs).

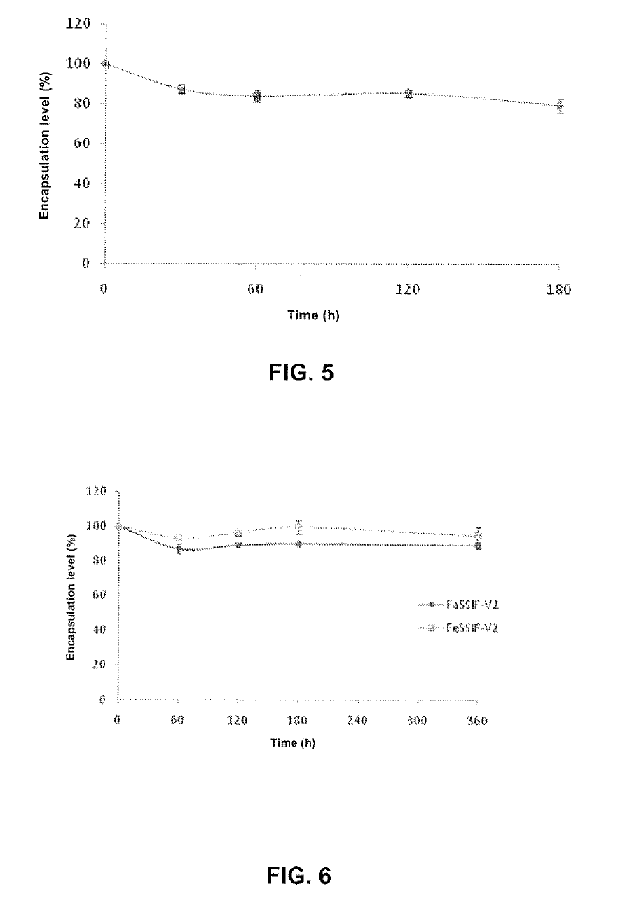

FIG. 5 illustrates the SN38 encapsulation level versus time with incubation of SN38-LNCs in a simulated gastric medium. 100% corresponds to the initial encapsulation level of the SN38 formulation.

FIG. 6 illustrates the SN38 encapsulation level versus time after incubation of SN38-LNCs in an empty intestinal medium or in a simulated fed intestinal medium. 100% corresponds to the initial encapsulation level of the SN38 formulation.

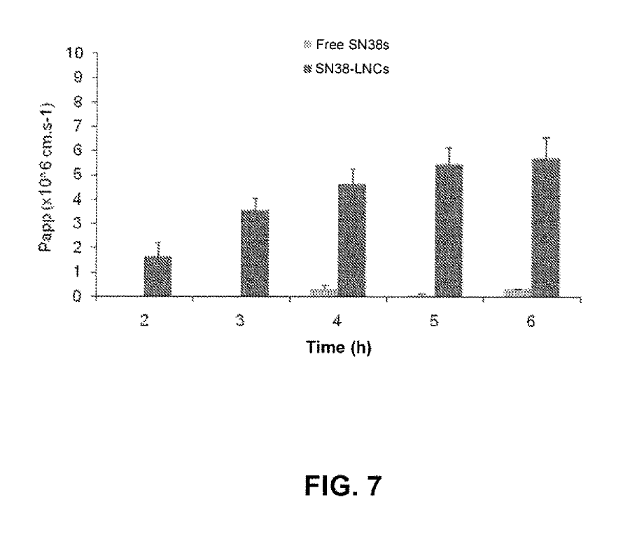

FIG. 7 illustrates the apparent permeability in cm·s⁻¹ versus time for the dispersion of free SN38 and of SN38 encapsulated in LNCs.

### EXAMPLE 1

### Lipid Nanocapsules not Loaded with an Active Ingredient (White LNCs)

### A) Preparation

7% w/w of Transcutol®HP, 9.8% w/w of Labrafil®M 1944 CS, 3.9% w/w of Labrafac® and 1.5% w/w Lipoid®S75-3 were mixed and heated to 85° C. for solubilizing the Lipoid®. After cooling, Solutol® HS15 (9.8% w/w), NaCl (1.0% w/w) and water (17.7%) were added and homogenized with magnetic stirring. Three gradual heating/cooling cycles between 65 and 90° C. were then carried out and at 70° C. during the last cycle, an irreversible shock was induced by dilution with water at 2° C. (49.3% w/w). Next, the suspension of LNCs was gently mixed with magnetic stirring for 5 mins at room temperature.

### B) Characterization

Table 1 below shows the average size of the nanocapsules obtained under the conditions described earlier, after three temperature cycles, their polydispersity and their zeta potential and the pH of the obtained dispersion.

TABLE I

| | Average | Polydispersity | Zeta | |
|---|---|---|---|---|
| | size (nm) | index (PDI) | potential (mV) | pH |
| White | 39 ± 3 | 0.210 ± 0.078 | −8 ± 1 | 7.4 ± 0.2 |
| LNCs | | | | |
| (n = 12) | | | | |

### EXAMPLE 2

### Lipid Nanocapsules Loaded with SN38

### A) Preparation

The SN38 was first of all solubilized in Transcutol®HP (0.5% w/w). To 7% w/w of this solution, 9.8% w/w of Labrafil®M 1944 CS, 3.9% w/w of Labrafac® and 1.5% w/w of Lipoid® S75-3 were added and the mixture was heated to 85° C. in order to solubilize the Lipoid®. After cooling, Solutol® HS15 (9.8% w/w), NaCl (1.0% w/w) and basic buffer (17.7%) were added and homogenized with magnetic stirring. The basic buffer was added in order to transform the free SN38 lactone into SN38 carboxylate. Three gradual heating/cooling cycles between 65 and 90° C. were then carried out and at 70° C. during the last cycle, an irreversible shock was induced by dilution with acid buffer at 2° C. (49.3% w/w). With this chill-hardening in an acid buffer, it is possible to transform SN38 back into the lactone form at the moment of its encapsulation. Next, the suspension of LNCs was gently mixed with magnetic stirring for 5 mins at room temperature.

### B) Characterization

Table II below shows the average size of the nanocapsules obtained under the conditions described earlier, after three temperature cycles, their polydispersity and their zeta potential and the pH of the obtained dispersion.

TABLE II

| | Average | Polydispersity | Zeta | |
|---|---|---|---|---|
| | size (nm) | index (PDI) | potential (mV) | pH |
| LNCs | 38 ± 2 | 0.133 ± 0.043 | −8 ± 1 | 7.4 ± 0.1 |
| loaded with | | | | |
| Sn38 (n = 30) | | | | |

FIG. 1 illustrates the time-dependent change in the conductivity versus the temperature of the oil/water emulsion described in Example 2, during the 3 temperature raising and lowering cycles between 50 and 95° C. The phase inversion zone begins at 70° C.; at this temperature the system is translucent. Therefore, the chill-hardening which causes the irreversible shock is carried out at a temperature <70° C., i.e. 68° C.

The encapsulation level of the SN38 in the LNCs was obtained by UV spectrometry after filtration. It is 0.38±0.06 mg/g of dispersion of LNCs, i.e. an encapsulation yield of 96±8% based on the initial amount weighed.

### EXAMPLE 3

### Formulation of Lipid Nanocapsules Loaded with SN38 in a Larger Volume

It is possible to formulate LNCs of SN38 by quadrupling the amounts.

### A) Preparation

The SN38 was first of all solubilized in Transcutol®HP (0.5% w/w). To 2.8 g of this solution, 4 g of Labrafil® M 1944 CS, 1.6 g of Labrafac® and 0.6 g of Lipoid® S75-3 were added and the mixture was heated to 85° C. in order to solubilize the Lipoid®. After cooling, Solutol® HS15 (4 g), NaCl (0.4 g), basic buffer (1.7 g) and water (7.2 g) were added and homogenized with magnetic stirring. Three gradual heating/cooling cycles between 65 and 90° C. were then carried out and at 70° C. for the last cycle, an irreversible shock was induced by dilution with acid buffer at 2° C. (20 g) by means of a syringe. Next, the suspension of LNCs was gently mixed with magnetic stirring for 5 mins at room temperature. A final amount of the dispersion of SN38 LNCs, of about 40 g, is obtained.

### B) Characterization

Table III below shows the average size of the nanocapsules obtained under the conditions described earlier, after three temperature cycles, their polydispersity and their zeta potential and the pH of the obtained dispersion.

TABLE III

| | | | Zeta | | Encapsulation |
|---|---|---|---|---|---|
| | Average | Polydispersity | potential | | level (mg/g of |
| | size (nm) | index (PDI) | Zeta (mV) | pH | dispersion) |
| LNCs | 35.1 ± 0.8 | 0.069 ± 0.014 | −8.7 ± 1.9 | 7.3 ± 0.1 | 0.45 ± 0.03 |
| Loaded with | | | | | |
| SN38 (n = 5) | | | | | |

### EXAMPLE 4

### Short Term Stability at Different pHs

A stability study was conducted over 6 h at 25° C. at 3 different pHs. The pH of the dispersion of the LNCs of SN38 was adjusted to 3, 7 or 10. The SN38 load level of the LNCs was measured after acidification of the sample taken with the purpose of precipitating SN38, and filtered with a Minisart® 0.2 μm filter (Sartorius, Göttingen, Germany).

FIG. 2 illustrates the SN38 encapsulation level versus time. 100% corresponds to the initial encapsulation level of the formulation of SN38.

At pH 3, rapid release of SN38 is observed. At pH 7, this release is less significant and is of the order of 40% after 6 h. At pH 10, the encapsulation level remains stable.

### EXAMPLE 5

### Long Term Stability at 4° C.

The stability of the LNCs loaded with SN38 was evaluated after storage at 2-8° C. The pH, the distribution of the particle sizes, the zeta potential and the SN38 load of the sample were determined after filtering the sample by using a Minisart® 0.2 μm filter (Sartorius, Götingen, Germany).

| | Encap- | Encap- | | | | |
|---|---|---|---|---|---|---|
| | sulation | sulation | | | | Zeta |
| Time | level | yield | Size | Poly- | | potential |
| (days) | (mg/g) | (%) | (nm) | dispersity | pH | (mV) |
| 0 | 0.43 ± | 89 ± 10 | 38.2 ± | 0.141 ± | 7.3 ± 0.1 | −7.7 ± 1.0 |
| | 0.06 | | 1.1 | 0.042 | | |
| 14 | 0.41 ± | 85 ± 13 | 39.0 ± | 0.147 ± | 7.6 ± 0.08 | −7.6 ± 0.3 |
| | 0.08 | | 1.1 | 0.055 | | |
| 28 | 0.34 ± | 72 ± 16 | 39.3 ± | 0.141 ± | 7.4 ± 0.03 | −8.5 ± 0.4 |
| | 0.04 | | 1.2 | 0.037 | | |
| 84 | 0.33 ± | 68 ± 12 | 40.1 ± | 0.135 ± | 7.5 ± 0.03 | −8.6 ± 0.7 |
| | 0.06 | | 1.1 | 0.037 | | |

At 4° C., there is no modification of the size, zeta potential and polydispersity of the nanocapsules. A 30% reduction in the encapsulation level is observed after 1 month.

### EXAMPLE 6

### Study of the Release of Encapsulated SN38

A study of the release of encapsulated SN38 in LNCs was carried out. The LNCs loaded with SN38 were diluted 1:200 (v/v) in saline phosphate buffer (PBS, pH=7.4) and placed at 37° C. with 150 rpm stirring. A 0.5 mL sample was taken and replaced with PBS at different time intervals. The samples were acidified and filtered using a Minisart® 0.2 μm filter in order to remove the precipitated free SN38. Next, the load was measured with LC-MS/MS. The release was calculated by difference with the initial load, and the profiles (release percentage versus time) were established.

FIG. 3 illustrates the release percentage of SN38 from the initial encapsulation level versus time.

The release percentage of the active ingredient of the LNCs at pH 7.4 is approximately 8% after 3 days.

### EXAMPLE 7

### Study of In Vitro Cytotoxicity of SN38-LNCs

The cytotoxicity of SN38-LNCs, of free SN38 and of unloaded LNCs was determined by an MTS test (CallTiter 96® AQuₑₒᵤₛ non-radioactive cell proliferation assay kit (Promega, Charbonnières, France)) on HT-29 cells (a human colorectal cancer line of human cells). The IC₅₀ was calculated as being the concentration of SN38-LNCs, of SN38 or unloaded LNCs causing 50% cell death.

FIG. 4 illustrates the cell survival percentage versus the concentration of SN38-LNCs, SN38 or unloaded LNCs (white LNCs). The obtained IC₅₀ with SN38 LNCs and with free SN38 is less than 0.1 μM. This result is approximately 250 times greater than the cytotoxicity obtained with CPT11.

### EXAMPLE 8

### Study of Gastro-Intestinal Stability

The stability of LNCs loaded with SN38 was evaluated in different simulated gastro-intestinal media at 37° C. with 150 rpm stirring. A 0.5 mL sample was analyzed at different time intervals. The samples were acidified and filtered by using a Minisart® 0.2 μm filter in order to remove the precipitated free SN38. Next, the SN38 load was measured by LC/MS/MS. The study was conducted on a simulated gastric medium described by the European Pharmacopeia (1), in a simulated empty intestinal medium and in a simulated fed intestinal medium (2).

FIG. 5 illustrates the SN38 encapsulation level versus time after incubation in a simulated gastric medium. 100% corresponds to the initial encapsulation level of the SN38 formulation.

FIG. 6 illustrates the SN38 encapsulation level versus time after incubation in a simulated empty intestinal medium or a simulated fed intestinal medium. 100% corresponds to the initial encapsulation level of the SN38 formulation.

### EXAMPLE 9

### Study of In Vitro Intestinal Permeability of the SN38-LNCs

The apparent permeability of free SN38 or SN38 encapsulated in LNCs was studied on an in vitro intestinal cell model (model Caco-2). The study is conducted on a system of culture chambers of the Transwell® type (Corning Costar, Cambridge, Mass.) at 37° C., 5% CO₂. A dispersion of free SN38 or of LNCs with SN38 at the concentration of 5 μM was deposited at the apical level of the culture chambers. Samples of 0.05 mL and of 0.150 mL were respectively taken at the apical and basolateral level respectively and replaced with HESS at different time intervals. The SN38 was measured by LC/MS/MS. The apparent permeability was measured according to the following formula (3, 4): Pₐₚₚ=dQ/dt×1/AC₀, (dQ/df=amount of SN38 at the basolateral level (μg·s⁻¹), C₀=initial SN38 concentration at the apical level (μg·mL⁻¹) and A=surface area of the cell monolayer (cm²).

FIG. 7 illustrates the apparent permeability in cm·s⁻¹ versus time for the dispersion of free SN38 and of encapsulated SN38 in LNCs. A Pₐₚₚ of 1.63±0.56.10⁶ cm·s⁻¹ is obtained after 6 h of incubation at 2 h and increases to 5.69±0.87.10⁶ cm·s⁻¹ at 6 h for the dispersion of SN38-LNCs. In the presence of free SN38, a Pₐₚₚ of 0.31±0.02.10⁶ cm·s⁻¹ is obtained after 6 h of incubation.

### REFERENCES

1. Pharmacopeia U. Rockville, Md.; 2006.
2. Jantratid E, Janssen N, Reppas C, Dressman J B. Dissolution media simulating conditions in the proximal human gastrointestinal tract: an update. Pharm. Res. 2008 July; 25(7):1663-76.
3. Artursson P, Borchardt R T. Intestinal drug absorption and metabolism in cell cultures: Caco-2 and beyond. Pharm. Res. 1997 December; 14(12):1655-8.
4. Artursson P, Karlsson J. Correlation between oral drug absorption in humans and apparent drug permeability coefficients in human intestinal epithelial (Caco-2) cells. Biochem. Biophys. Res. Commun. 1991 Mar. 29; 175(3):880-5.

1. Nanocapsules comprising:
a core essentially consisting of a liquid or semi-liquid fat at room temperature, and comprising a hydrophobic active ingredient and a diethylene glycol ether,
an external lipid shell, solid at room temperature.

2. The nanocapsules according to claim 1, wherein the diethylene glycol ether is diethylene glycol monoethyl ether.

3. The nanocapsules according to claim 1, wherein the active ingredient is SN38.

4. The nanocapsules according to claim 3, wherein they contain more than 0.3 mg of SN38 per gram of nanocapsules.

5. The nanocapsules according to claim 1, wherein the fat of the core essentially consists in at least one triglyceride, a fatty acid ester, a polyethoxylene glyceride, or one of their mixtures.

6. The nanocapsules according to claim 5, wherein the triglyceride is a C₈-C₁₂ triglyceride.

7. The nanocapsules according to claim 5, wherein the polyethoxylene glyceride is a PEG-6 ester of apricot kernel oil.

8. The nanocapsules according to claim 1, wherein the external shell essentially consists in a lipophilic surfactant and a hydrophilic surfactant.

9. The nanocapsules according to claim 8, wherein the lipophilic surfactant is a phospholipid, for which the proportion of phosphotidylcholine varies from 40 to 90% by weight.

10. The nanocapsules according to claim 8, wherein the non-ionic hydrophilic surfactant is a polyethylene glycol 2-hydroxystearate.

11. The nanocapsules according to claim 1, wherein the lipids/[active ingredient+diethylene glycol ether] core ratio is comprised between 0.5:1 and 1:2.

12. The nanocapsules according to claim 1, comprising
a core consisting of SN38, of diethylene glycol monoethylether, of a capric and caprylic triglyceride and a PEG-6 ester of apricot kernel oil,
a shell consisting of lecithin for which the proportion of phosphatidylcholine is comprised between 40% and 90%, and of polyethylene glycol 2-hydroxystearate is in a ratio from 1:0.09 to 0.15:1.

13. The nanocapsules according to claim 1, wherein said core consists in
a central core consisting of the hydrophobic active ingredient and of a diethylene glycol ether,
a lipid layer surrounding said nucleus.

14. A method for preparing nanocapsules comprising the following steps:
a) solubilizing an active ingredient in a solution of a diethylene glycol ether,
b) preparing an oil/water emulsion by adding to the solution of step a) at least one triglyceride, one polyethoxylene glyceride, one lipophilic surfactant solid at 20° C., one non-ionic hydrophilic surfactant and a salt,
c) achieving phase inversion of said oil/water emulsion by increasing the phase inversion temperature (PIT) with stirring, in order to obtain a water/oil emulsion, followed by a decrease in the temperature down to a temperature T1, T1<PIT<T2,
d) carrying out one or more temperature cycles with stirring around the phase inversion zone between T1 and T2, until a translucent suspension is observed,
e) achieving chill-hardening with an acid aqueous solution of the oil/water emulsion at a temperature close to T1, preferably greater than T1, in order to obtain stable nanocapsules.

15. The preparation method according to claim 14, wherein step b) is broken down as follows:
b1) adding to the solution of step a) at least one triglyceride, a polyethoxylene glyceride and a lipophilic surfactant,
b2) heating until solubilization of the lipophilic surfactant,
b3) cooling,
b4) adding the hydrophilic surfactant and salt.

16. The preparation method according to claim 14, wherein:
the active ingredient is SN38,
a basic buffer is added in step b4) for transforming the SN38 as a free lactone into SN38 as a carboxylate,
the chill-hardening is achieved by dilution in step e) with an acid buffer at 2° C.±1° C.

17. The method according to claim 14, wherein the oil/water emulsion contains:
1 to 3% of lipophilic surfactant
5 to 15% of hydrophilic surfactant
5 to 15% of oily fat
5 to 10% of diethylene glycol ether
40 to 65% of water,
the percentages being expressed by weight.

18. The method according to claim 14, wherein the aqueous phase of the oil/water emulsion further contains 1 to 4% of a salt.

19. The use of nanocapsules according to claim 1, for making a drug administered via an oral, sublingual, subcutaneous, intramuscular, intravenous, intrathecal, epidural, transdermal, local or rectal route.

20. The nanocapsules of claim 6 wherein the triglyceride is selected from capric and caprylic acid triglyceride.

21. A method of treatment of cancer according to claim 1, comprising a step of administering an effective amount of nanocapsules to a patient in need thereof.
